(19) United States
(12) Patent Application Publication
(10) Pub. No.: US 2026/0224173 A1
(43) Pub. Date: Aug. 6, 2026

(54) BIOLOGICAL INFORMATION MEASUREMENT DEVICE, CONTROL METHOD OF BIOLOGICAL INFORMATION MEASUREMENT DEVICE, AND STORAGE MEDIUM

(71) Applicant: OMRON HEALTHCARE CO., LTD., Muko-shi (JP)

(72) Inventors: Kentaro Mori, Kyoto (JP); Tsuyoshi Kitagawa, Kyoto (JP)

(73) Assignee: OMRON HEALTHCARE CO., LTD., Muko-shi (JP)

(21) Appl. No.: 19/445,852

(22) Filed: Jan. 12, 2026

(30) Foreign Application Priority Data
Feb. 5, 2025 (JP) 2025-017923

(51) Int. Cl. A61B 5/00; A61B 5/022

(52) U.S. Cl. CPC A61B 5/7282; A61B 5/02225; A61B 5/7435

(57) ABSTRACT

A biological information measurement device including: a blood pressure measurement unit that acquires information for measuring blood pressure; an arrhythmia information acquisition unit that acquires information related to determination of presence or absence of arrhythmia during measurement of the blood pressure; a display unit; an input unit; a storage unit; and a control unit, wherein the control unit calculates a blood pressure value on the basis of the information acquired by the blood pressure measurement unit, and causes, in a case where the information acquired by the arrhythmia information acquisition unit indicates that arrhythmia during the measurement of the blood pressure has been present, the display unit to display contrast information enabling recognition of a difference between the blood pressure value calculated and a normal blood pressure value.

### CROSS-REFERENCE TO RELATED APPLICATIONS

The disclosure of Japanese Patent Application No.JP 2025-017923, filed on February 5, 2025 including the specification, drawings and abstract is incorporated herein by reference in its entirety.

### TECHNICAL FIELD

The present invention relates to a biological information measurement device capable of measuring blood pressure of a living body, a control method of a biological information measurement device, and a program.

### BACKGROUND ART

In recent years, it has become widespread to perform health management by measuring information regarding the body and health of an individual such as a blood pressure value with a measuring instrument and recording and analyzing the measurement result. In particular, since arrhythmia such as atrial fibrillation (AF) may lead to a brain/cardiovascular disease, it is desirable that the user himself/herself can easily recognize such an abnormality.

In this regard, for example, there has been proposed a sphygmomanometer that displays, in a case where atrial fibrillation has been detected at the time of blood pressure measurement, the fact that there has been atrial fibrillation together with a blood pressure measurement result (for example, Patent Document 1). According to such a sphygmomanometer, the user can quickly recognize the presence of arrhythmia while performing daily health management of blood pressure measurement.

Furthermore, Patent Document 2 that discloses a biological information acquisition device discloses that, in a case where arrhythmia has been detected in an electrocardiogram test performed prior to blood pressure measurement, notifying the detection of arrhythmia makes it possible to notify the user of a decrease in reliability of the blood pressure measurement.

### PRIOR ART DOCUMENTS

### Patent Documents

Patent Document 1: JP 2024-039189 A

Patent Document 2: JP 2008-168074 A

### SUMMARY

However, if the blood pressure value is displayed as the measurement result even in a case where arrhythmia occurs, the user may focus on the displayed blood pressure value and overlook the occurrence of the arrhythmia. In the blood pressure measurement result when arrhythmia has been detected, the blood pressure value cannot have been accurately measured and is inaccurate. Therefore, the user erroneously recognizes the inaccurate blood pressure value as a result of appropriately measuring the blood pressure value in a case where the information regarding the occurrence of arrhythmia is overlooked.

In view of the above circumstances, an object of the present invention is to provide a technology capable of preventing a user from erroneously recognizing an inaccurate blood pressure value of the time when the arrhythmia has been detected as an accurate blood pressure value and of notifying of the arrhythmia detected during blood pressure measurement.

In order to solve the above problem, the present invention adopts the following configuration. The configuration is

a biological information measurement device including:

a blood pressure measurement unit that acquires information for measuring blood pressure of a living body;

an arrhythmia information acquisition unit that acquires information related to determination of presence or absence of arrhythmia in the living body during measurement of the blood pressure;

a display unit including an area capable of displaying at least the blood pressure;

an input unit that receives an operation input by a user;

a storage unit that stores information related to measurement of the blood pressure performed in a past; and

a control unit, wherein

the control unit

calculates a blood pressure value on the basis of the information acquired by the blood pressure measurement unit, and

causes, in a case where the information acquired by the arrhythmia information acquisition unit indicates that arrhythmia during the measurement of the blood pressure has been present, the display unit to display contrast information enabling recognition of a difference between the blood pressure value calculated and a normal blood pressure value.

Note that the information related to the presence or absence of arrhythmia acquired by the arrhythmia information acquisition unit may be information on the presence or absence of arrhythmia detected by another biological information measurement device or a biological signal itself measured by another device. Examples of “arrhythmia” include atrial fibrillation, atrial flutter, and premature contraction.

Furthermore, the “contrast information” may be an average value of the blood pressure of the living body measured when arrhythmia is absent, or may be a value indicating a difference between the blood pressure value calculated and the average value of the blood pressure of the living body measured when arrhythmia is absent.

According to such a configuration, in a case where arrhythmia has been present during the blood pressure measurement, not only the blood pressure value calculated as the measurement result but also the contrast information enabling recognition of the difference from the normal blood pressure value (calculated when arrhythmia is absent) are displayed together. Therefore, it is possible to prevent the user from erroneously recognizing the blood pressure value of the time when arrhythmia has been detected as an accurate blood pressure value and to notify of the arrhythmia detected during the blood pressure measurement.

Furthermore, the control unit may

receive, via the input unit, a request to display, on the display unit, the information related to the measurement of the blood pressure performed in the past, the information being held in the storage unit, and

in a case where the information related to the measurement of the blood pressure related to the request to display includes information of a fact that arrhythmia has been present during the measurement, cause the display unit to display a past value of the blood pressure related to the request to display and the contrast information.

With such a configuration, the user can view the past measurement history, and even in a case where arrhythmia has been detected at the time of the past measurement, it is possible to prevent the user who has viewed the measurement result from erroneously recognizing the blood pressure value of the time when the arrhythmia has been detected as the accurate blood pressure value and to recognize the arrhythmia detected during the blood pressure measurement.

Furthermore, the control unit may detect body motion during the measurement of the blood pressure on the basis of the information acquired by the blood pressure measurement unit, and may cause, in a case where the number of times of detection of the body motion during the measurement of the blood pressure is equal to or more than a predetermined value, the display unit to display that the number of times of detection of the body motion during the measurement of the blood pressure is equal to or more than the predetermined value. With such a configuration, in a case body motion during measurement is frequent, it is possible to indicate to the user the possibility that the accuracy of the measured blood pressure value has decreased.

Furthermore, the arrhythmia information acquisition unit may acquire the information related to presence or absence of arrhythmia in the living body on the basis of pulse wave information acquired by the blood pressure measurement unit. For example, in a case where the blood pressure measurement unit calculates the blood pressure on the basis of the pressure pulse wave, arrhythmia can be detected on the basis of the pressure pulse wave.

Furthermore, the biological information measurement device may further include an electrocardiographic waveform acquisition unit that acquires an electrocardiographic waveform of the living body, wherein the arrhythmia information acquisition unit may acquire the information related to presence or absence of arrhythmia in the living body on the basis of the electrocardiographic waveform during the measurement of the blood pressure.

Furthermore, the present invention can also be regarded as a method as follows. The method is

a control method of a biological information measurement device, including:

measuring blood pressure of a living body;

determining presence or absence of arrhythmia in the living body during measurement of the blood pressure; and

displaying a measurement result of the blood pressure, wherein,

in a case where it is determined, in the determining presence or absence of arrhythmia, that arrhythmia has been present, contrast information enabling recognition of a difference between a measured value of the blood pressure and a normal blood pressure value is displayed in the displaying the measurement result.

Furthermore, the present invention can also be regarded as a program for causing the biological information measurement device to execute the above-described control method, and a computer-readable recording medium in which such a program is non-transiently recorded.

Note that each of configurations and processing described above can be combined with each other to constitute the present invention as long as no technical contradiction occurs.

According to the present invention, it is possible to prevent a user from erroneously recognizing an inaccurate blood pressure value of the time when arrhythmia has been detected as an accurate blood pressure value and to notify of the arrhythmia detected during blood pressure measurement.

### BRIEF DESCRIPTION OF THE DRAWINGS

FIG. 1 is a block diagram schematically illustrating a device configuration of a biological information measurement device according to a first embodiment;

FIG. 2 is a block diagram illustrating functional modules implemented by a control unit of the biological information measurement device according to the first embodiment;

FIG. 3 is a flowchart illustrating a flow of blood pressure measurement processing by the biological information measurement device according to the first embodiment;

FIG. 4A is a first diagram illustrating an example of a measurement result display screen when arrhythmia has been detected during blood pressure measurement;

FIG. 4B is a second diagram illustrating an example of the measurement result display screen when arrhythmia has been detected during blood pressure measurement;

FIG. 5 is a flowchart illustrating a flow of processing when a history viewing mode is executed in the biological information measurement device according to the first embodiment;

FIG. 6 is a block diagram schematically illustrating a device configuration of a biological information measurement device according to a second embodiment;

FIG. 7 is a block diagram illustrating functional modules implemented by a control unit of the biological information measurement device according to the second embodiment;

FIG. 8 is a block diagram schematically illustrating a device configuration of a biological information measurement device according to a third embodiment;

FIG. 9 is a block diagram illustrating functional modules implemented by a control unit of the biological information measurement device according to the third embodiment;

FIG. 10 is a block diagram schematically illustrating an information processing system according to a fourth embodiment;

FIG. 11A is a block diagram illustrating functional modules implemented by a control unit of a biological information processing device according to the fourth embodiment;

FIG. 11B is a block diagram illustrating functional modules implemented by a control unit of an information processing terminal according to the fourth embodiment;

FIG. 12 is a first flowchart illustrating a flow of processing of each of the biological information measurement device and the information processing terminal in the information processing system according to the fourth embodiment; and

FIG. 13 is a second flowchart illustrating a flow of processing of each of the biological information measurement device and the information processing terminal in the information processing system according to the fourth embodiment.

### DESCRIPTION OF THE EMBODIMENTS

### First embodiment

Hereinafter, specific embodiments of the present invention will be described on the basis of the drawings. However, the specific detail of each configuration described in the embodiments described below, the arrangement relationship thereof, and the like are not intended to limit the scope of the present invention only to them unless otherwise specified.

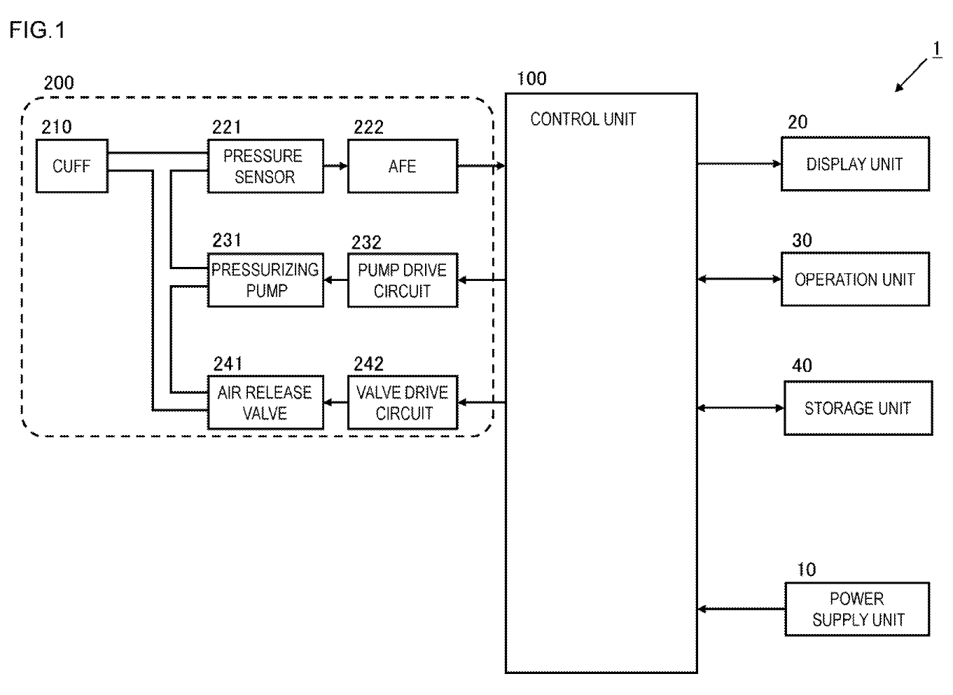

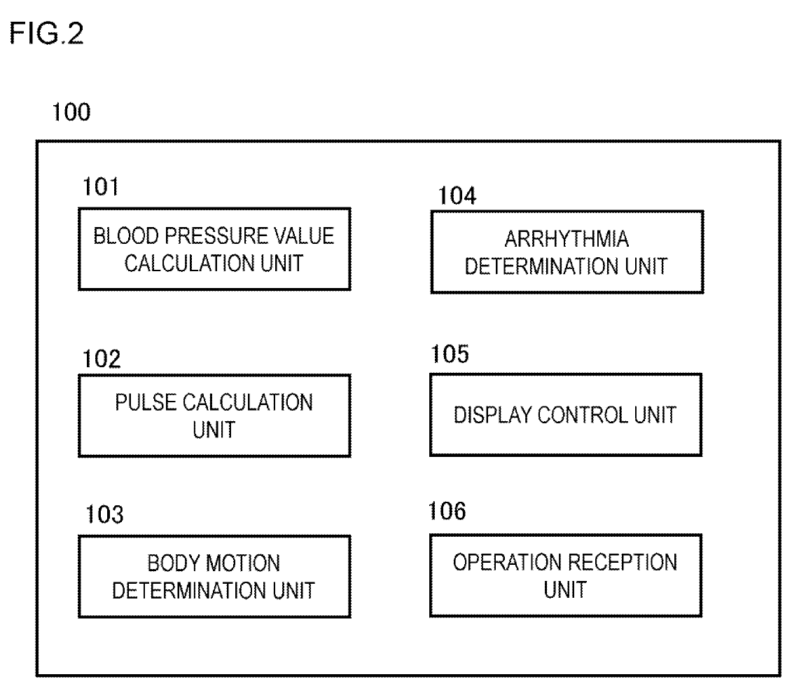

The present invention can be applied to, for example, a biological information measurement device 1 as illustrated in FIGS. 1 and 2. FIG. 1 is a block diagram schematically illustrating a device configuration of the biological information measurement device 1 in the present embodiment, and FIG. 2 is a block diagram illustrating an example of functional modules implemented by a control unit 100 of the biological information measurement device 1.

As illustrated in FIG. 1, the biological information measurement device 1 according to the present embodiment schematically includes components such as a power supply unit 10, a display unit 20, an operation unit 30, a storage unit 40, the control unit 100, and a blood pressure measurement unit 200, and has a configuration similar to that of a general portable upper-arm sphygmomanometer including a main body housing, a cuff, and an air tube as an appearance although not illustrated.

The power supply unit 10 is a power source of the biological information measurement device 1, and may be, for example, a rechargeable secondary battery such as a lithium ion battery, but is not limited thereto, and may be a primary battery, a terminal connectable to an external power supply, or the like.

The display unit 20 is a functional unit for displaying various types of information, and can be, for example, a display device such as a liquid crystal display (LCD). Furthermore, the operation unit 30 is a functional unit that receives a user operation such as turning on/off of a power supply, execution of measurement, and selection/decision of a menu, and can be configured by an operation switch and the like. Note that a touch panel display can also be adopted as a configuration that serves as both the display unit 20 and the operation unit 30.

The storage unit 40 includes a main storage device such as a random access memory (RAM) and a read only memory (ROM), and an auxiliary storage device such as an HDD and a flash memory, and stores various types of information such as an application program, measurement results such as a blood pressure value and a pulse, information on the presence or absence of arrhythmia to be described later, and other acquired biological information. Note that the measurement results of the blood pressure and the like and other acquired biological information may be stored in the storage unit 40 in association with time information such as the measurement (acquisition) time. As the time information, for example, time information clocked by referring to a real time clock (RTC) of the control unit 100 to be described later can be used. Furthermore, the auxiliary storage device may be configured to be detachable from the main body housing.

The control unit 100 is a functional unit that controls the entire biological information measurement device 1, and includes, for example, a processor such as a central processing unit (CPU). When receiving a user's operation via the operation unit 30, the control unit 100 controls each component of the biological information measurement device 1 to execute various processing such as blood pressure measurement and presentation of various types of information according to a predetermined program. The predetermined program is stored in the storage unit 40 and read therefrom. As the processor, in addition to the CPU, a graphic processing unit (GPU), a field programmable grid array (FPGA), an application specific integrated circuit (ASIC), or the like can be adopted.

Furthermore, functional modules illustrated in FIG. 2, including a blood pressure value calculation unit 101, a pulse calculation unit 102, a body motion determination unit 103, an arrhythmia determination unit 104, a display control unit 105, and an operation reception unit 106 are implemented by the control unit 100. These functional modules will be described in detail later.

The blood pressure measurement unit 200 includes a cuff 210, a pressure sensor 221, an analog front end (AFE) 222 including an analog-to-digital (A/D) converter, a pressurizing pump 231, a pump drive circuit 232, an air release valve 241, and a valve drive circuit 242, and is a functional unit that measures the blood pressure of the user by a so-called oscillometric method. The blood pressure measurement by the oscillometric method is a well-known technology, and thus a detailed description thereof will be omitted.

The cuff 210 is a member used with being wound around the upper arm of the user and includes the pressurizing pump 231 (pump drive circuit 232) disposed in the main body housing, an air bladder (not illustrated) communicating with the air release valve 241 (valve drive circuit 242) via an air tube (not illustrated), a belt (not illustrated) incorporating the air bladder, the pressure sensor 221 provided on the belt, and the like.

Note that the belt of the cuff 210 is provided with a fixing unit (for example, a hook-and-loop fastener) for fixing the cuff 210 to the upper arm of the user, and the cuff 210 is wound around the upper arm of the user by the belt when blood pressure measurement is performed using the biological information measurement device 1.

For example, a piezoresistive sensor including a piezoelectric element can be adopted as the pressure sensor 221, and at least a pressure pulse wave of the user is acquired. In addition, the biological information measurement device 1 according to the present embodiment calculates the blood pressure and the pulse of the user on the basis of the pressure pulse wave acquired by the pressure sensor 221, and detects the presence or absence of the body motion and arrhythmia of the user during the blood pressure measurement as described later.

At the time of blood pressure measurement, the control unit 100 controls the pressurizing pump 231 (pump drive circuit 232) and the air release valve 241 (valve drive circuit 242) to adjust the cuff pressure of the cuff 210. Specifically, control is performed such that the pressurizing pump 231 is driven to send air to the cuff 210 to inflate the cuff 210 (increase the cuff pressure). In this way, after the blood flow is once inhibited by compressing the blood vessel of the upper arm of the user, control is performed such that the pressurizing pump 231 is stopped to open the air release valve 241 and the air is gradually released from the cuff 210 to contract the cuff 210 (reduce the cuff pressure).

Next, functional modules illustrated in FIG. 2 will be described. The blood pressure value calculation unit 101 calculates the blood pressure value of the user on the basis of the pressure pulse wave acquired by the pressure sensor 221. Note that, in the present embodiment, the blood pressure value is calculated by the oscillometric method, but it is also possible to provide a microphone in the cuff 210 and calculate the blood pressure value by, for example, a Korotkoff method of detecting a Korotkoff sound.

The pulse calculation unit 102 calculates the pulse rate of the user on the basis of the pressure pulse wave (such as the peak number of the pulse wave) acquired by the pressure sensor 221. The body motion determination unit 103 determines the presence or absence and the number of times of body motion during blood pressure measurement on the basis of the pressure pulse wave (such as disturbance of pulse waveform) acquired by the pressure sensor 221.

Furthermore, the arrhythmia determination unit 104 determines (detects) the presence or absence of arrhythmia during the blood pressure measurement on the basis of the pressure pulse wave (information on the time interval between the peaks of the pulse wave, and the like) acquired by the pressure sensor 221. That is, in the present embodiment, a combination of the arrhythmia determination unit 104 and the pressure sensor 221 corresponds to an arrhythmia information acquisition unit.

The display control unit 105 controls the display unit 20 (the display contents thereof). For example, the display control unit 105 causes the display unit 20 to display menu selection, measurement results, past history data, and the like, and specific display contents of the measurement results will be described later.

The operation reception unit 106 receives an operation input by a user via the operation unit 30. For example, when the user presses a measurement start button, an item selection button, a decision button, or the like (each including an interface expressed on an image), the biological information measurement device 1 is controlled to perform the associated operation.

### Flow of blood pressure measurement processing

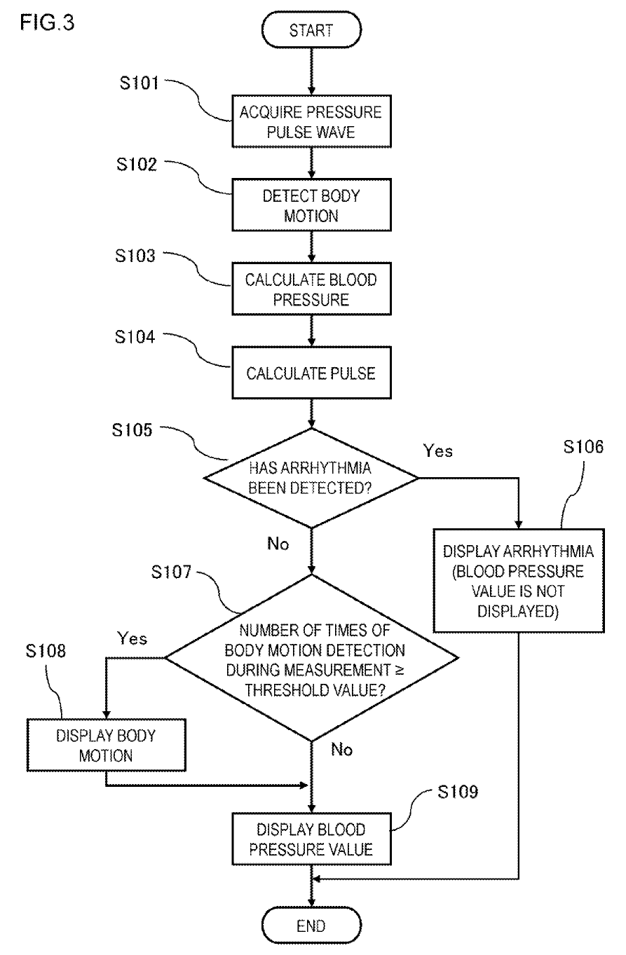

Next, a flow of processing when measurement of blood pressure is executed by the biological information measurement device 1 will be described on the basis of FIG. 3. FIG. 3 is a flowchart illustrating a flow of processing by the biological information measurement device 1 when measurement of the blood pressure is performed. Prior to the execution of the blood pressure measurement, the user winds the cuff 210 around his/her upper arm, and then instructs execution of the blood pressure measurement via the operation unit 30.

After receiving the instruction of the blood pressure measurement, the biological information measurement device 1 acquires the pressure pulse wave of the user by the blood pressure measurement unit 200 (S101). Based on the acquired pressure pulse wave, the control unit 100 (the blood pressure value calculation unit 101, the pulse calculation unit 102, and the body motion determination unit 103) detects the body motion of the user (S102), calculates the blood pressure value (S103), and calculates the pulse rate (S104). In addition, the control unit 100 (arrhythmia determination unit 104) determines the presence or absence of arrhythmia during blood pressure measurement on the basis of the acquired pressure pulse wave (S105).

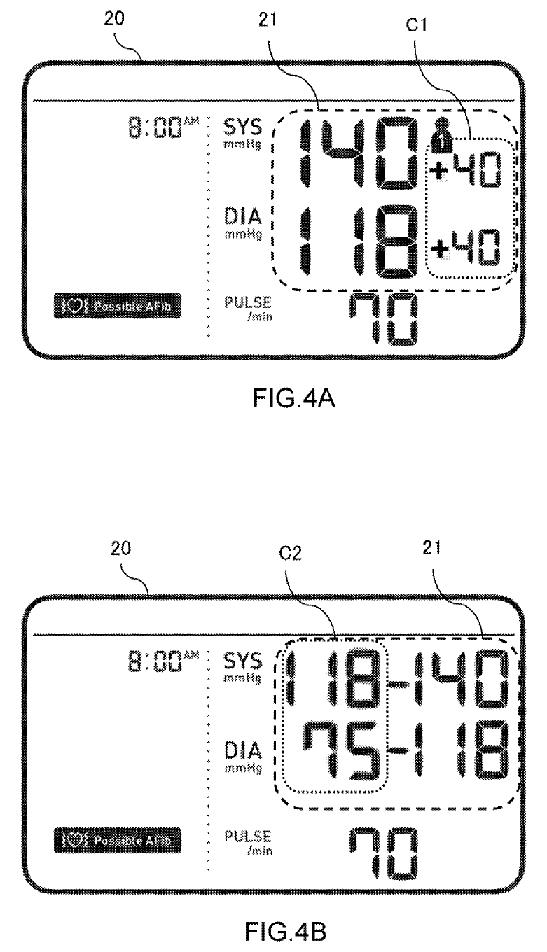

In a case where it is determined in step S105 that arrhythmia has been present during the blood pressure measurement, the control unit 100 (display control unit 105) displays not only the calculated blood pressure value (inaccurate blood pressure value) but also the contrast information enabling recognition of the difference from the normal blood pressure value on the display unit 20 (S106). FIG. 4A is a diagram illustrating an example of the display unit 20 when displaying the measurement result of a case where arrhythmia has been detected during the blood pressure measurement in this manner.

In the example illustrated in FIG. 4A, in a blood pressure display area 21 for displaying the calculated blood pressure value of the display unit 20, the calculated blood pressure value is displayed on the left side, and contrast information C1 is displayed on the right side thereof, the contrast information C1 indicating a value of a difference from the average value of the blood pressure measured when arrhythmia has not been detected (hereinafter, referred to as normal-time blood pressure). Furthermore, a mark indicating that atrial fibrillation has been detected is also displayed in a blinking manner on the lower left side of the screen. Note that the average value of the normal-time blood pressure can be acquired, for example, by obtaining an average of all the blood pressure data of the cases where arrhythmia has not been detected at the time of measurement among the past measurement data stored in the storage unit 40 or an average of N times (for example, 10 times) from the latest measurement.

On the other hand, in a case where it is determined in step S105 that arrhythmia has been absent during the blood pressure measurement, the control unit 100 (body motion determination unit 103) determines whether or not the number of times of body motion detected during the blood pressure measurement exceeds a predetermined threshold value (S107). In a case where it is determined in step S107 that the number of times of body motion is equal to or less than the threshold value, the control unit 100 (display control unit 105) displays the blood pressure value in the blood pressure display area 21 of the display unit 20 (S109).

In a case where it is determined in step S107 that the number of times of body motion exceeds the threshold value, the control unit 100 (display control unit 105) displays the fact that body motion has been detected (which may result in the low accuracy of the blood pressure value) in an area other than the blood pressure display area 21 of the display unit 20 (S108), and displays the blood pressure value in the blood pressure display area 21 (S109). Note that step S108 and step S109 may be executed simultaneously.

After displaying the blood pressure measurement result on the display unit 20 in step S106 or step S109, the biological information measurement device 1 stores the measurement result in the storage unit 40 in association with the information on the measurement time, and ends the series of processing.

With the display of the measurement result as described above, in a case where arrhythmia has been detected during the blood pressure measurement, it is possible to prevent the user from erroneously recognizing an inaccurate blood pressure value as an accurate blood pressure value and to notify the user of the arrhythmia detected during the blood pressure measurement. Furthermore, even in a case where arrhythmia has not been detected, when the number of times of body motion exceeds the threshold value, it is possible to alert the user by displaying that the accuracy of the calculated blood pressure value has decreased together with the blood pressure value.

### Flow of past data display processing

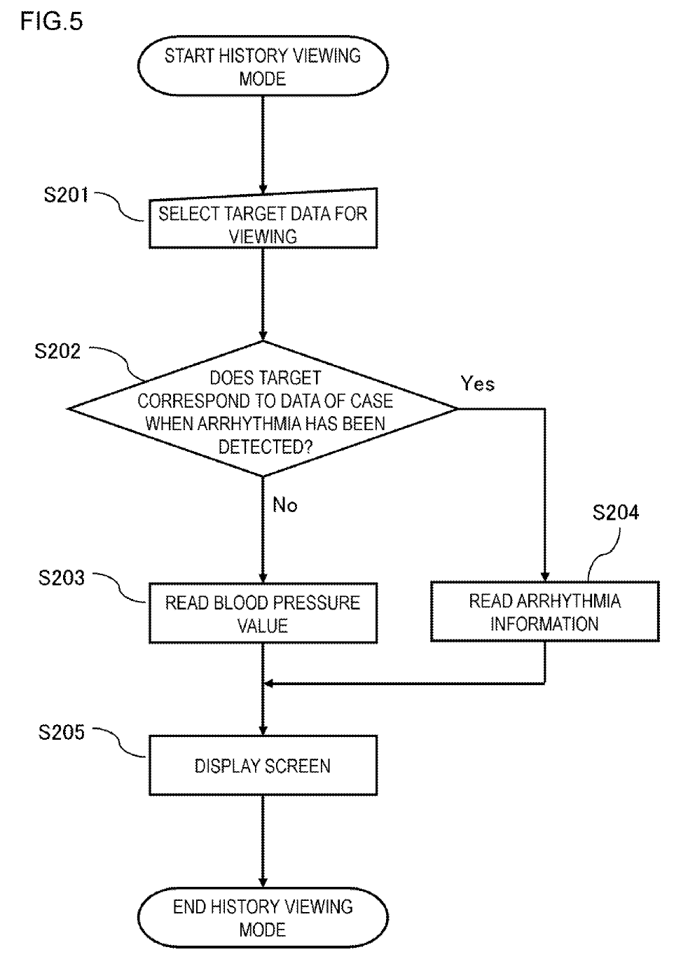

Subsequently, a flow of processing when the past measurement data stored in the storage unit 40 is displayed on the display unit 20 will be described on the basis of FIG. 5. FIG. 5 is a flowchart illustrating a flow of processing (history viewing mode) of referring to past measurement data in the biological information measurement device 1. As illustrated in FIG. 5, first, past measurement data (for example, which is read from the storage unit 40 and displayed as a list on the display unit 20) that the user wants to refer to is selected via the operation unit 30 (S201).

Next, the control unit 100 determines whether or not the measurement data according to the user's selection is data of the case where arrhythmia has been detected during the measurement (S202). Specifically, since the measurement data of the case where arrhythmia has been detected is stored in association with the fact that arrhythmia has been detected and the detail of the arrhythmia, it is determined whether or not the selected measurement data corresponds to such data. In a case where it is determined in step S202 that the measurement data according to the selection is not data of the case where arrhythmia has been detected during the measurement, the control unit 100 reads information on the measured blood pressure value (S203), displays the information on the display unit 20 (S205), and ends the series of processing.

On the other hand, in a case where it is determined in step S202 that the measurement data according to the selection is measurement data of the case where arrhythmia has been detected during the measurement, the control unit 100 reads the information on the arrhythmia (S204), displays a measurement result screen (screen as illustrated in FIG. 4A) indicating the measured blood pressure value and the contrast information in combination on the display unit 20 (S205), and ends the series of processing.

According to such processing, the user can view the past measurement result, and even in a case where arrhythmia has been detected at the time of the past measurement, it is possible to prevent the user from erroneously recognizing an inaccurate blood pressure value as an accurate blood pressure value, and to notify the user of the arrhythmia detected during the blood pressure measurement.

### Modification

In the above embodiment, the contrast information displayed together with the blood pressure value in a case where arrhythmia has been detected during the blood pressure measurement is the value indicating the difference between the blood pressure value of the time when arrhythmia has been detected and the average value of the normal-time blood pressure, but the contrast information may be other information. FIG. 4B is a diagram illustrating a display screen example according to such a modification. In the diagram illustrated in FIG. 4B, in the blood pressure display area 21, the calculated blood pressure value is displayed on the right side, and on the left side thereof, contrast information C2 indicating the average value of the normal-time blood pressure is displayed. That is, in this modification, the display is performed in a mode of contrasting the blood pressure value calculated when arrhythmia has been detected with the average value of the normal-time blood pressure. As a result, the user can confirm the average blood pressure in normal times and easily visually recognize the difference from the current blood pressure value compared with the average blood pressure.

Furthermore, the contrast information may be a general “proper value” according to the attribute of the user (for example, age, sex, and the like), a difference from the proper value, or the like.

### Second embodiment

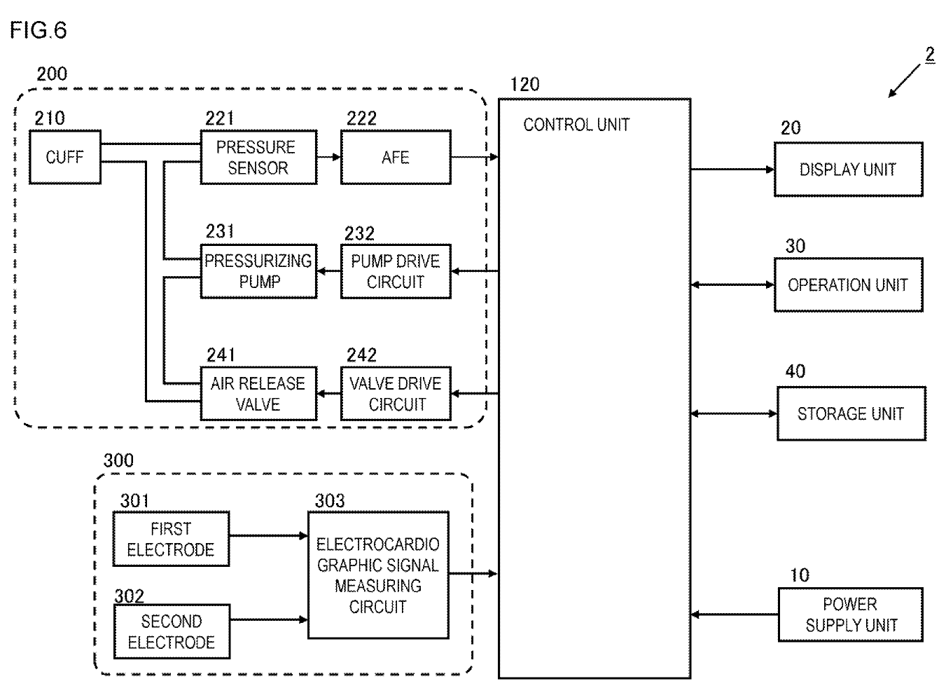

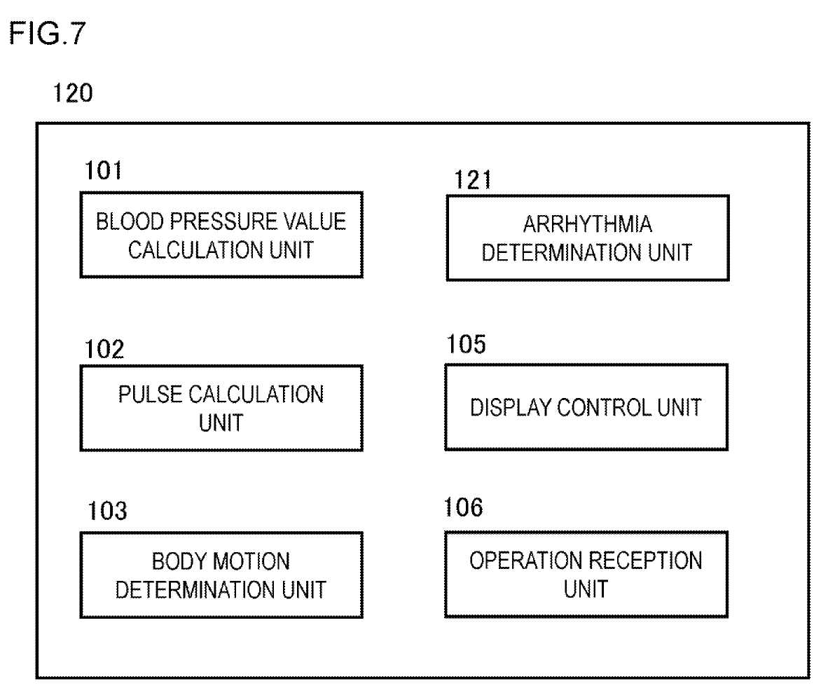

Next, another embodiment of the present invention will be described on the basis of FIGS. 6 and 7. A biological information measurement device 2 according to the present embodiment is a hybrid biological information measurement device capable of measuring not only blood pressure but also an electrocardiographic waveform. Note that, since the biological information measurement device 2 includes many configurations common to those of the biological information measurement device 1 of the first embodiment, hereinafter, the same reference numerals and signs are used for the same configurations as those of the first embodiment, and repeated description will be omitted.

As illustrated in FIG. 6, the biological information measurement device 2 includes an electrocardiographic waveform measurement unit 300 in addition to the same configurations as those of the biological information measurement device 1. The electrocardiographic waveform measurement unit 300 includes a plurality of electrodes (a first electrode 301 and a second electrode 302) and an electrocardiographic signal measuring circuit 303. The electrocardiographic signal measuring circuit 303 is a circuit for measuring an electrocardiographic waveform on the basis of an electrocardiographic signal obtained on the basis of a potential difference between two electrodes in contact with a living body, and includes an amplifier, a filter, an A/D conversion circuit, and the like.

Although not illustrated, the first electrode 301 and the second electrode 302 are disposed at positions separated from each other in the main body housing. When the user performs measurement processing in a state where one of the left and right fingers is in contact with the first electrode 301 and the other is in contact with the second electrode 302, the potential difference between the first electrode 301 and the second electrode 302 is measured as an electrocardiographic waveform by the electrocardiographic signal measuring circuit 303 (so-called I induction), and the electrocardiographic waveform (electrocardiogram) is measured by recording the potential difference.

Furthermore, as illustrated in FIG. 7, the biological information measurement device 2 includes functional modules including the blood pressure value calculation unit 101, the pulse calculation unit 102, the body motion determination unit 103, the display control unit 105, and an arrhythmia determination unit 121 which are implemented by a control unit 120.

In the present embodiment, the arrhythmia determination unit 121 detects arrhythmia on the basis of not the pressure pulse wave but the electrocardiographic waveform measured by the electrocardiographic signal measuring circuit 303. Specifically, by simultaneously performing the blood pressure measurement by the blood pressure measurement unit 200 and the electrocardiographic waveform measurement by the electrocardiographic waveform measurement unit 300, the arrhythmia determination unit 121 determines the presence or absence of arrhythmia during the blood pressure measurement on the basis of the electrocardiographic waveform acquired during the blood pressure measurement. That is, in the present embodiment, a combination of the arrhythmia determination unit 121 and the electrocardiographic waveform measurement unit 300 corresponds to the arrhythmia information acquisition unit. Furthermore, the display control unit 105 causes the display unit 20 to display a graph display of the acquired electrocardiographic waveform in response to a user operation.

Note that the flow of processing of the blood pressure measurement in the biological information measurement device 2 according to the present embodiment is similar to that of the biological information measurement device 1 of the first embodiment except that the detection of arrhythmia is performed on the basis of the electrocardiographic waveform instead of the pressure pulse wave, and the display contents of the blood pressure measurement result, the viewing of the past history data of the blood pressure values, and the like are also similar to those of the biological information measurement device 1.

As in the biological information measurement device 2 according to the present embodiment, by detecting arrhythmia on the basis of the electrocardiographic waveform, it is possible to more accurately detect arrhythmia during the blood pressure measurement.

### Third embodiment

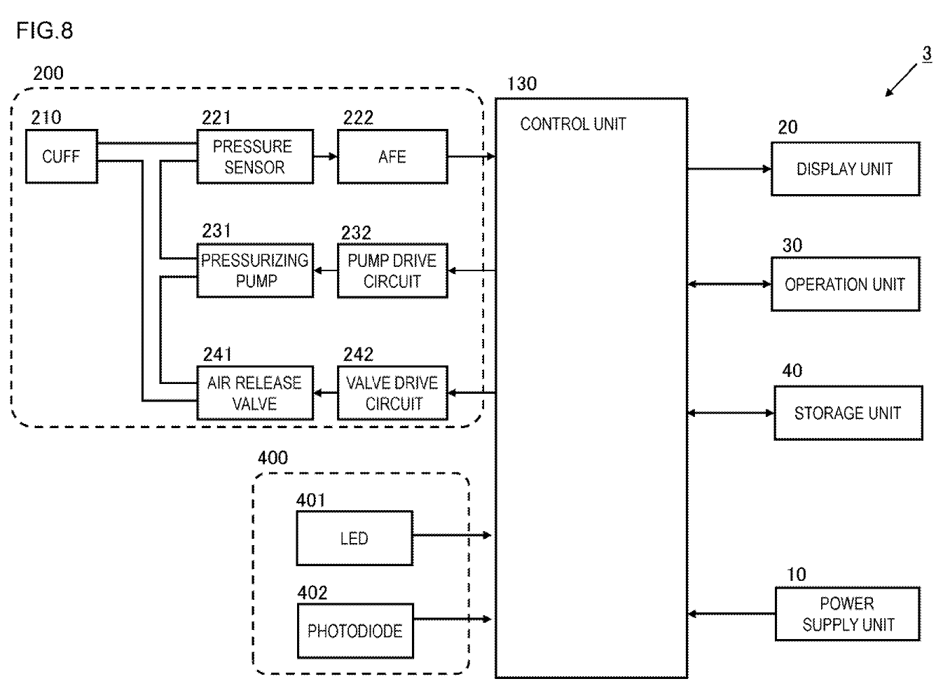

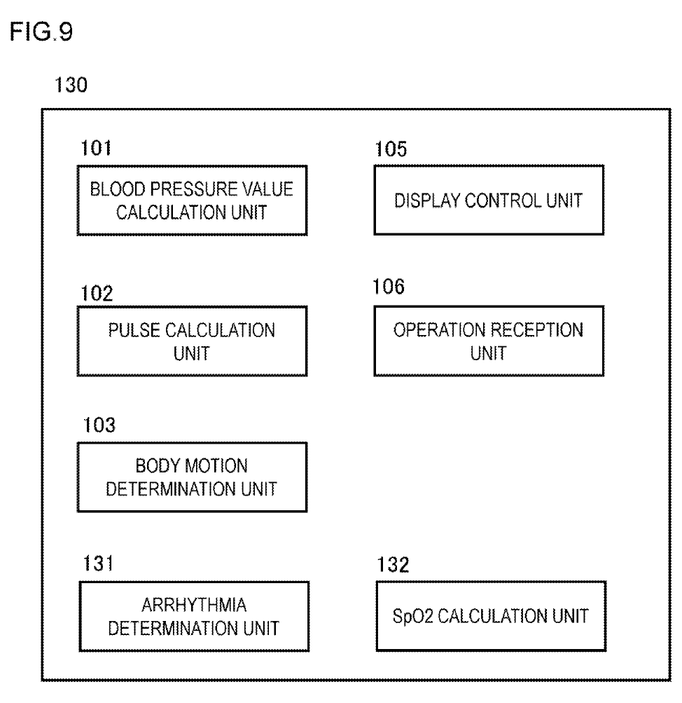

Subsequently, still another embodiment of the present invention will be described on the basis of FIGS. 8 and 9. A biological information measurement device 3 according to the present embodiment is a hybrid biological information measurement device capable of measuring not only blood pressure but also blood oxygen saturation (SpO2).

As illustrated in FIG. 8, the biological information measurement device 3 includes an SpO2 measurement unit 400 in addition to the same configurations as those of the biological information measurement device 1. The SpO2 measurement unit 400 includes an LED 401 as a light emitting element and a photodiode 402 as a light receiving element, and acquires information for calculating blood oxygen saturation by receiving reflected light of red light or infrared light emitted from the LED 401 by the photodiode 402.

Note that the SpO2 measurement unit 400 can not only measure the blood oxygen saturation but also detect a change in the blood flow rate accompanying the heartbeat as a change in the reflected light received by the photodiode 402 and acquire the pulse wave on the basis of the change.

Furthermore, as illustrated in FIG. 9, the biological information measurement device 3 includes functional modules including the blood pressure value calculation unit 101, the pulse calculation unit 102, the body motion determination unit 103, the display control unit 105, an arrhythmia determination unit 131, and an SpO2 calculation unit 132 which are implemented by a control unit 130.

The SpO2 calculation unit 132 calculates the blood oxygen concentration using the information acquired by the SpO2 measurement unit 400 (the intensity of the reflected light received by the photodiode 402).

Furthermore, in the present embodiment, the arrhythmia determination unit 131 detects arrhythmia on the basis of not the pressure pulse wave but the photoelectric pulse wave acquired by the SpO2 measurement unit 400. Specifically, by simultaneously performing the blood pressure measurement by the blood pressure measurement unit 200 and the measurement by the SpO2 measurement unit 400, the arrhythmia determination unit 131 determines the presence or absence of arrhythmia during the blood pressure measurement on the basis of the pulse wave information acquired by the SpO2 measurement unit 400. That is, in the present embodiment, a combination of the arrhythmia determination unit 131 and the SpO2 measurement unit 400 corresponds to the arrhythmia information acquisition unit.

Note that the flow of processing of the blood pressure measurement in the biological information measurement device 3 according to the present embodiment is similar to that of the biological information measurement device 1 of the first embodiment except that the detection of arrhythmia is performed on the basis of the photoelectric pulse wave instead of the pressure pulse wave, and the display contents of the blood pressure measurement result, the viewing of the past history data of the blood pressure values, and the like are also similar to those of the biological information measurement device 1.

### Fourth embodiment

### System configuration

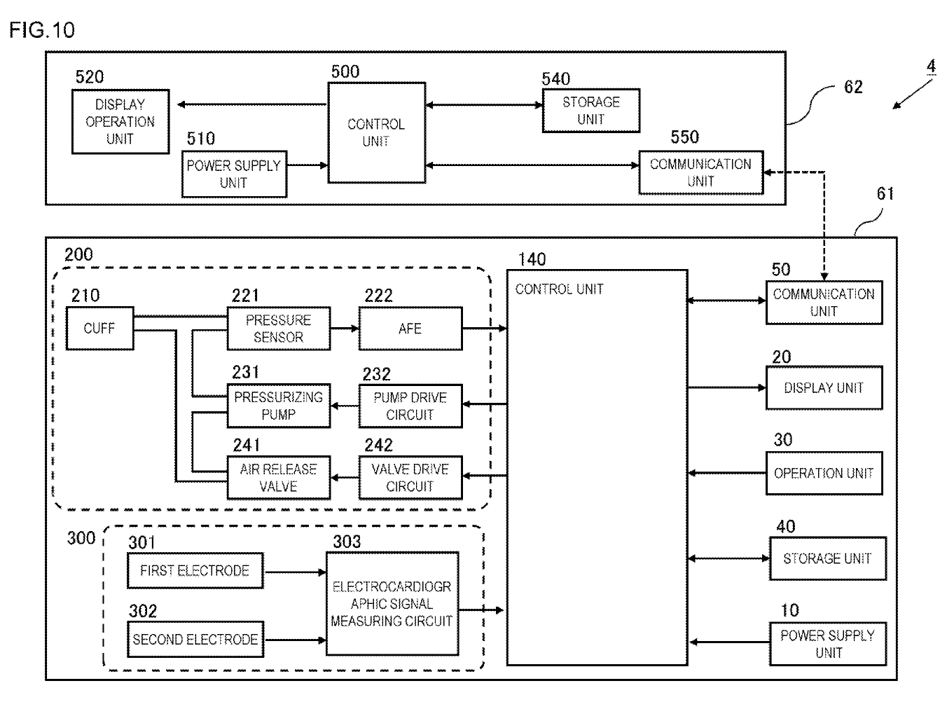

In each of the above embodiments, the detection of arrhythmia during the blood pressure measurement (determination of the presence or absence of arrhythmia) is executed in the biological information measurement device, but the detection of arrhythmia during the blood pressure measurement may be executed in another device. That is, the present invention is also applicable as a system including a biological information measurement device and an information processing terminal as in an embodiment described below. FIG. 10 is a block diagram schematically illustrating a configuration of an information processing system 4 according to the present embodiment.

As illustrated in FIG. 10, the information processing system 4 according to the present embodiment includes a biological information measurement device 61 including the blood pressure measurement unit 200 and the electrocardiographic waveform measurement unit 300, and a smartphone 62 as an example of the information processing terminal, which are configured to be communicably connectable.

The biological information measurement device 61 has a hardware configuration substantially similar to that of the biological information measurement device 2 according to the second embodiment, and is only different in including a communication unit 50. The communication unit 50 functions to communicate with and connect to the smartphone 62, and can adopt an appropriate communication interface according to a communication network to be connected, such as a wired communication port or a wireless communication antenna. Note that a communication method between the biological information measurement device 61 and the smartphone 62 is not particularly limited, and for example, pairing by Bluetooth (registered trademark) low energy (BLE) communication, communication by Wi-Fi (registered trademark) (LAN), or the like can be adopted.

As illustrated in FIG. 10, the smartphone 62 includes configurations such as a power supply unit 510, a display operation unit 520, a storage unit 540, a communication unit 550, and a control unit 500.

The power supply unit 510 is a power source of the smartphone 62, and can be, for example, a rechargeable secondary battery such as a lithium ion battery. The display operation unit 520 can be a touch panel display or the like having both functions of information display and input operation reception.

The storage unit 540 includes a main storage device such as a RAM and a ROM, and an auxiliary storage device such as a flash memory, and stores various types of information such as an application program and various pieces of biological information. Note that the measurement results of the blood pressure and the like and other acquired biological information may be stored in the storage unit 540 in association with time information such as the measurement (acquisition) time. As the time information, for example, time information clocked by referring to an RTC of the control unit 500 can be used. Furthermore, the auxiliary storage device may be configured to be detachable from the main body housing (not illustrated).

The communication unit 550 functions to communicate with and connect to the biological information measurement device 61, and can adopt an appropriate communication interface according to a communication network to be connected, such as a wired communication port or a wireless communication antenna.

The control unit 500 is a functional unit that controls the smartphone, and includes, for example, a processor such as a CPU and a GPU. When receiving a user's operation via the display operation unit 520, the control unit 500 controls each component of the smartphone to exhibit various functions according to a predetermined application program. Note that the predetermined program is stored in the storage unit 540 and read therefrom.

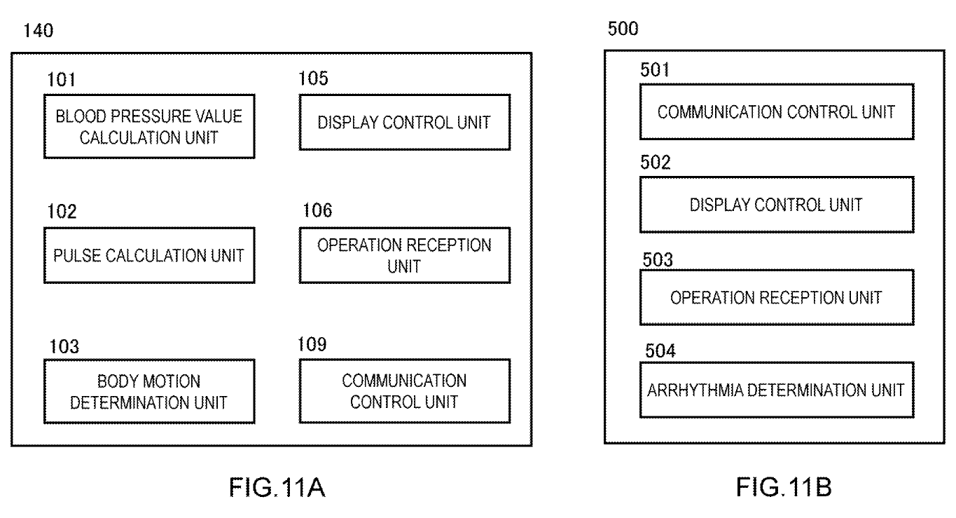

FIG. 11A is a block diagram illustrating part of functional modules implemented by a control unit 140 of the biological information measurement device 61, and FIG. 11B is a block diagram illustrating part of functional modules implemented by the control unit 500 of the smartphone 62.

As illustrated in FIG. 11A, the functional modules of the biological information measurement device 61 are different from those of the biological information measurement device 2 of the second embodiment in that a communication control unit 109 is added and the arrhythmia determination unit does not exist. The communication control unit 109 executes communication with an external device (for example, the smartphone 62) by the communication unit 50.

As illustrated in FIG. 11B, the control unit 500 of the smartphone 62 implements functional modules including a communication control unit 501, a display control unit 502, and an arrhythmia determination unit 504. The communication control unit 109 executes communication with an external device (for example, the biological information measurement device 61) by the communication unit 50.

The display control unit 502 controls the display contents of the display operation unit 520. For example, the display control unit 502 causes the display operation unit 520 to display a menu screen (operation reception screen), guidance related to the progress of biological information measurement, a measurement result of biological information, and the like according to an application program related to cooperation with the biological information measurement device 61.

The operation reception unit 503 receives an input of a user operation via a user interface displayed on the display operation unit 520. For example, in accordance with an application program related to cooperation with the biological information measurement device 61, the operation reception unit 503 receives an operation input such as start of measurement, selection of an item, and screen transition by the biological information measurement device 61.

As will be described later, the arrhythmia determination unit 504 determines (detects), on the basis of the information on the electrocardiographic waveform transmitted from the biological information measurement device 61, the presence or absence of arrhythmia at the time of blood pressure measurement performed simultaneously with the acquisition of the electrocardiographic waveform. That is, in the present embodiment, the detection processing of arrhythmia during the blood pressure measurement is performed not by the biological information measurement device 61 but by the smartphone 62. Then, the information on the presence or absence of arrhythmia determined by the arrhythmia determination unit 504 is transmitted from the communication unit 550 and received by the communication unit 50 of the biological information measurement device 61. That is, in the biological information measurement device 61 in the present embodiment, the communication unit 50 corresponds to the arrhythmia information acquisition unit.

### Flow of processing

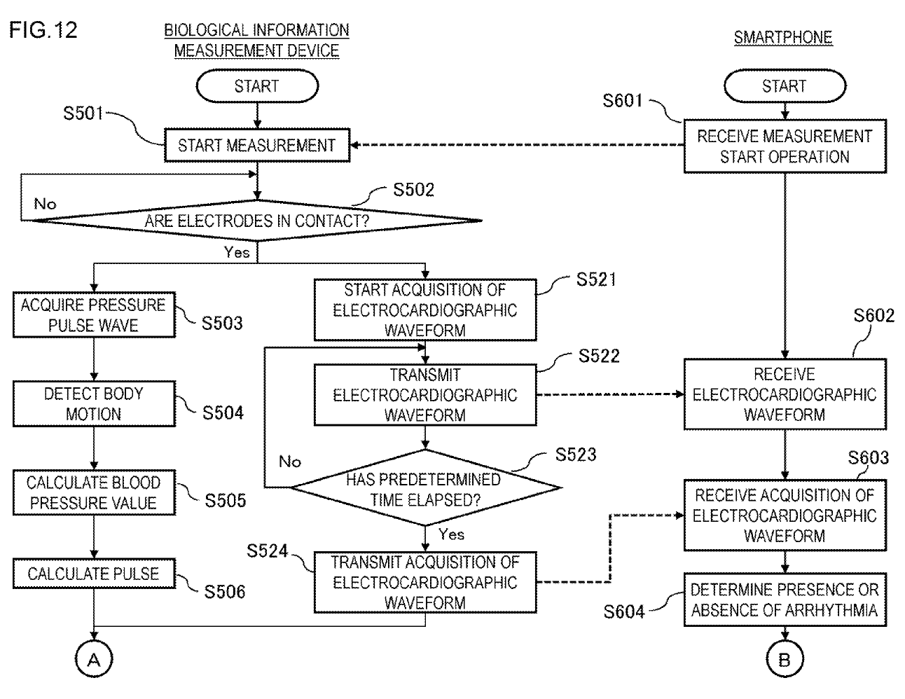

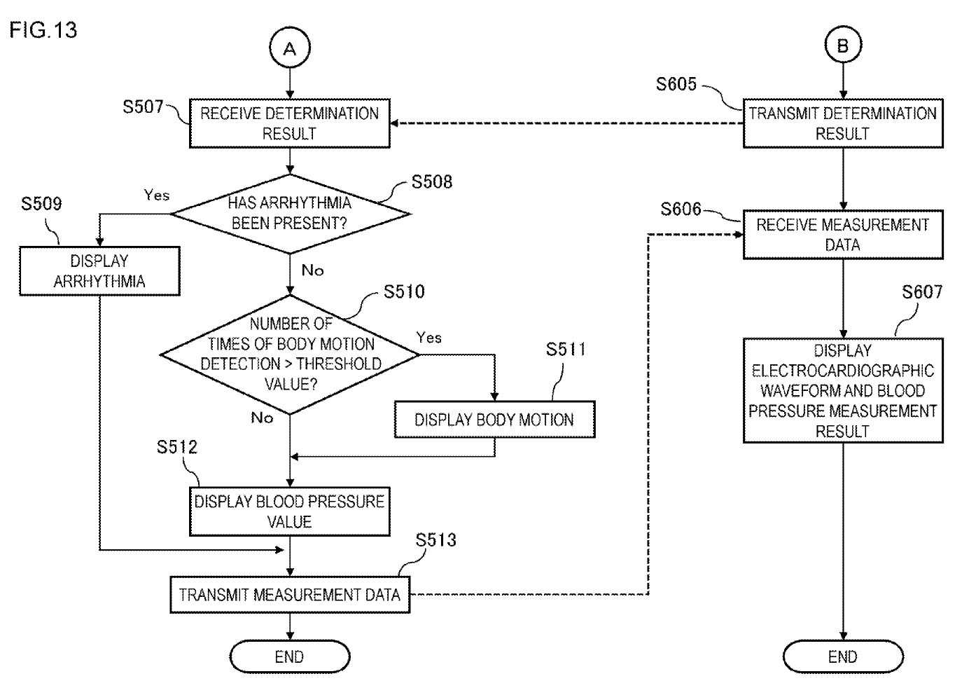

Subsequently, based on FIGS. 12 and 13, flows of blood pressure/electrocardiogram collective measurement processing executed in the information processing system 4 according to the present embodiment and information will be described. In the information processing system 4 according to the present embodiment, the smartphone 62 can instruct to start the collective measurement of the blood pressure and the electrocardiographic waveform. Therefore, the control unit 500 (operation reception unit 503) of the smartphone 62 receives the operation input of starting the collective measurement of the blood pressure and the electrocardiogram via the display operation unit 520 (S601). The instruction by the operation input is transmitted from the smartphone 62 to the biological information measurement device 61 via the communication unit 550, and the biological information measurement device 61 starts the collective measurement of the blood pressure and the electrocardiogram (S501).

When the collective measurement is started, the biological information measurement device 61 determines whether or not the first electrode 201 and the second electrode 202 are correctly in contact with the living body on the basis of the signal acquired by the electrocardiographic waveform measurement unit 300 (S502). Here, in a case where it is determined that both electrodes are not correctly in contact with the living body, the processing to determine the electrode contact is repeatedly performed. On the other hand, in a case where it is determined in step S502 that both electrodes are correctly in contact with the living body, the biological information measurement device 61 executes acquisition of the pressure pulse wave by the blood pressure measurement unit 200 and acquisition of the electrocardiographic waveform by the electrocardiographic waveform measurement unit 300 in parallel (S503, S521).

The electrocardiographic waveform acquired in step S521 by the biological information measurement device 61 is transmitted to the smartphone 62 via the communication unit 50 (S522), and the smartphone 62 receives the electrocardiographic waveform (S602). The biological information measurement device 61 (control unit 140) continues to acquire the electrocardiographic waveform, and determines whether or not a predetermined time (for example, time sufficient for pulse wave acquisition for blood pressure measurement) has elapsed (S523). Here, in a case where it is determined that the predetermined time has not elapsed, the process returns to step S522, and the subsequent processing is repeated.

On the other hand, in a case where it is determined in step S523 that the predetermined time has elapsed, the biological information measurement device 61 ends the acquisition of the electrocardiographic waveform and transmits that fact to the smartphone 62 via the communication unit 50 (S524), and the smartphone 62 receives the information of the fact that the acquisition of the electrocardiographic waveform has ended (S603). Then, in the smartphone 62 that has received the electrocardiographic waveform for the predetermined time, the control unit 500 (arrhythmia determination unit 504) executes analysis processing of determining (detecting) the presence or absence of arrhythmia during the blood pressure measurement (during pressure pulse wave acquisition) on the basis of the electrocardiographic waveform (S604).

On the other hand, in the biological information measurement device 61, in parallel with the acquisition of the electrocardiographic waveform, acquisition of a pressure pulse wave for blood pressure measurement is performed by the blood pressure measurement unit 200 (S503). In addition, based on the acquired pressure pulse wave, the control unit 140 (the blood pressure value calculation unit 101, the pulse calculation unit 102, and the body motion determination unit 103) detects the body motion of the user (S504), calculates the blood pressure value (S505), and calculates the pulse rate (S506).

Next, referring to FIG. 13, the smartphone 62 transmits the determination result of the presence or absence of arrhythmia performed in step S604 to the biological information measurement device 61 via the communication unit 550 (S605), and the biological information measurement device 61 receives the determination result (S507). Then, in a case where the determination result received in step S507 is the presence of arrhythmia during the blood pressure measurement, the biological information measurement device 61 (display control unit 105) displays the calculated blood pressure value and the contrast information together on the display unit 20 (S508, S509). The contents of the contrast information are as described above.

On the other hand, in a case where the received determination result is the absence of arrhythmia during the blood pressure measurement, the biological information measurement device 61 (body motion determination unit 103) subsequently determines whether or not the number of times of body motion detected during the blood pressure measurement exceeds a predetermined threshold value (S508, S510). Then, in a case where it is determined in step S510 that the number of times of body motion is equal to or less than the threshold value, the biological information measurement device 61 (display control unit 105) displays the blood pressure value and the pulse rate on the display unit 20 (S512).

In a case where it is determined in step S510 that the number of times of body motion exceeds the threshold value, the biological information measurement device 61 (display control unit 105) displays the fact that body motion has been detected in an area other than the blood pressure display area 21 of the display unit 20 (S511), and displays the blood pressure value in the blood pressure display area 21 (S512). Note that step S511 and step S512 may be executed simultaneously.

Thereafter, the biological information measurement device 61 transmits the calculated blood pressure value, pulse rate, and number of times of body motion detection (hereinafter, collectively referred to as blood pressure measurement data) to the smartphone 62 via the communication unit 50 (S513), and ends the series of processing. On the other hand, the smartphone 62 receives the blood pressure measurement data (S606), displays the blood pressure measurement data and the electrocardiographic waveform (electrocardiogram) on the display operation unit 520 (S607), and ends the series of processing.

Note that the order of the processing described above can be appropriately changed or omitted, and for example, the transmission of the blood pressure measurement data from the biological information measurement device 61 is not limited to after step S512, and may be executed at other timings such as before or after step S507. Furthermore, the smartphone 62 may end the series of processing after step S605 without transmitting the blood pressure measurement data from the biological information measurement device 61.

Furthermore, since it is undetermined which of the processing of the blood pressure value calculation and the processing of the arrhythmia presence or absence determination ends first, step S507 (and step S605) may be executed before the blood pressure value calculation in step S505. In the smartphone 62, after the electrocardiographic waveform is received in step S602, the electrocardiographic waveform may be displayed on the display operation unit 520 in real time.

In the above example, in step S523, the biological information measurement device 61 determines whether or not the predetermined time has elapsed from the start of acquisition of the electrocardiographic waveform. However, this processing may be executed on the smartphone 62 side. In this case, the smartphone 62 may transmit a command to end the acquisition of the electrocardiographic waveform to the biological information measurement device 61 after the predetermined time elapses.

According to the information processing system 4 as described above, the determination of the presence or absence of arrhythmia during the blood pressure measurement can be executed by the information processing terminal such as the smartphone 62 instead of the biological information measurement device 61. As a result, the calculation load of the biological information measurement device can be reduced, and even if a high-performance calculation device is not used, it is possible to prevent an inaccurate blood pressure measurement result from being indicated to the user and to notify the user of the arrhythmia detected during the blood pressure measurement.

### Others

Note that the description of the above-described embodiments is merely illustrative of the present invention, and the present invention is not limited to the above-described specific forms. The present invention can be variously modified and combined within the scope of the technical idea. For example, the configuration according to the second embodiment and the configuration according to the third embodiment may be combined to form a biological information measurement device including all of the blood pressure measurement unit 200, the electrocardiographic waveform measurement unit 300, and the SpO2 measurement unit 400.

In the above embodiments, when the fact that arrhythmia has been detected during the blood pressure measurement is displayed, the display mode may be made different from the display mode at the time of notifying of another measurement error or failure of the device. Specifically, the detection of arrhythmia can be clearly identified from other errors by a difference in lighting/blinking of the display, a difference in blinking interval, a difference in display color, and the like.

In the second and third embodiments, the electrocardiographic waveform measurement unit 300 and the SpO2 measurement unit 400 are exemplified as the devices integrated with the blood pressure measurement unit 200. However, the present invention is not limited thereto, and a configuration can be adopted in which the electrocardiographic waveform and the photoelectric pulse wave acquired by a separate measuring instrument are acquired via the communication unit. In this case, the communication unit serves as the arrhythmia information acquisition unit. Furthermore, the present invention can also be applied to a wearable type, such as a wristwatch type, biological information measurement device.

In the fourth embodiment, the biological information measurement device 61 includes the blood pressure measurement unit 200 and the electrocardiographic waveform measurement unit 300. However, the technology of determining the presence or absence of arrhythmia during the blood pressure measurement using another information processing terminal is naturally applicable to other biological information measurement devices as described in the first and third embodiments. Furthermore, the information processing terminal is not limited to the smartphone 62, and other computers can be adopted regardless of whether the computer is a portable computer or a stationary computer.

In each of the above examples, the description has been made using the atrial fibrillation as an example of arrhythmia. However, as a matter of course, also in a case where another arrhythmia (atrial flutter, premature contraction, etc.) has been detected, the fact that atrial flutter or premature contraction has been detected may be displayed without displaying the blood pressure value similarly to the above embodiments.

What is claimed is:

1. A biological information measurement device comprising:
a blood pressure measurement unit that acquires information for measuring blood pressure of a living body;
an arrhythmia information acquisition unit that acquires information related to determination of presence or absence of arrhythmia in the living body during measurement of the blood pressure;
a display unit including an area capable of displaying at least the blood pressure;
an input unit that receives an operation input by a user;
a storage unit that stores information related to measurement of the blood pressure performed in a past; and
a control unit, wherein
the control unit
calculates a blood pressure value on the basis of the information acquired by the blood pressure measurement unit, and
causes, in a case where the information acquired by the arrhythmia information acquisition unit indicates that arrhythmia during the measurement of the blood pressure has been present, the display unit to display contrast information enabling recognition of a difference between the blood pressure value calculated and a normal blood pressure value.

2. The biological information measurement device according to claim 1, wherein
the contrast information is an average value of the blood pressure of the living body measured when the arrhythmia is absent.

3. The biological information measurement device according to claim 1, wherein
the contrast information is a value of a difference between the blood pressure value calculated and an average value of the blood pressure of the living body measured when the arrhythmia is absent.

4. The biological information measurement device according to claim 1, wherein
the control unit
receives, via the input unit, a request to display, on the display unit, the information related to the measurement of the blood pressure performed in the past, the information being held in the storage unit, and
causes, in a case where the information related to the measurement of the blood pressure related to the request to display includes information of a fact that arrhythmia has been present during the measurement, the display unit to display a past value of the blood pressure related to the request to display and the contrast information.

5. The biological information measurement device according to claim 1, wherein
the control unit
detects body motion during the measurement of the blood pressure on the basis of the information acquired by the blood pressure measurement unit, and
causes, in a case where the number of times of detection of the body motion during the measurement of the blood pressure is equal to or more than a predetermined value, the display unit to display that the number of times of detection of the body motion during the measurement of the blood pressure is equal to or more than the predetermined value.

6. The biological information measurement device according to claim 1, wherein
the arrhythmia information acquisition unit acquires the information related to presence or absence of arrhythmia in the living body on the basis of pulse wave information acquired by the blood pressure measurement unit.

7. The biological information measurement device according to claim 1, further comprising
an electrocardiographic waveform acquisition unit that acquires an electrocardiographic waveform of the living body, wherein
the arrhythmia information acquisition unit acquires the information related to presence or absence of arrhythmia in the living body on the basis of the electrocardiographic waveform during the measurement of the blood pressure.

8. A control method of a biological information measurement device, comprising:
measuring blood pressure of a living body;
determining presence or absence of arrhythmia in the living body during measurement of the blood pressure; and
displaying a measurement result of the blood pressure, wherein,
in a case where it is determined, in the determining presence or absence of arrhythmia, that arrhythmia has been present, contrast information enabling recognition of a difference between a measured value of the blood pressure and a normal blood pressure value is displayed in the displaying the measurement result.

9. A non-transitory computer-readable storage medium recording a program for causing a biological information measurement device to execute the measuring, the determining, and the displaying according to claim 8.
